(19) United States
(12) Patent Application Publication
(10) Pub. No.: US 2026/0053735 A1
(43) Pub. Date: Feb. 26, 2026

(54) OCULAR HYDROGEL TYROSINE KINASE INHIBITOR IMPLANTS

(71) Applicant: Ocular Therapeutix, Inc., Bedford, MA (US)

(72) Inventors: Peter Jarrett, Burlington, MA (US); Rami El-Hayek; Timothy S. Jarrett, Boston, MA (US); Zachary Lattrell, Newburyport, MA (US); Arthur Driscoll, Reading, MA (US); Charles D. Blizzard, Nashua, NH (US)

(21) Appl. No.: 19/373,051

(22) Filed: Oct. 29, 2025

Related U.S. Application Data
(63) Continuation of application No. 18/583,383, filed on Feb. 21, 2024.
Application No. 18/583,383 is a continuation of application No. 17/880,175, filed on Aug. 3, 2022, now abandoned.
Application No. 17/880,175 is a continuation of application No. 16/857,463, filed on Apr. 24, 2020, now abandoned.
(60) Provisional application No. 62/838,796, filed on Apr. 25, 2019.
(60) Provisional application No. 62/838,998, filed on Apr. 26, 2019.
(60) Provisional application No. 62/994,391, filed on Mar. 25, 2020.

(51) Int. Cl. A61K 9/00; A61K 31/4439

(52) U.S. Cl. CPC A61K 9/0024; A61K 9/0051; A61K 31/4439

(57) ABSTRACT

Provided herein are sustained-release biodegradable ocular hydrogel implants which are useful in the treatment of certain ocular conditions.

### RELATED APPLICATIONS

This application claims priority to U.S. Provisional Application No. 62/838,796, filed Apr. 25, 2019, U.S. Provisional Application No. 62/838,998, filed Apr. 26, 2019, and U.S. Provisional Application No. 62/994,391, filed Mar. 25, 2020, the entire contents of each of which are incorporated herein by reference.

### BACKGROUND

Macular degeneration, also called age-related macular degeneration (AMD) is the leading cause of vision loss, affecting more than ten million Americans. AMD is caused by the deterioration of the central portion of the retina, the macula, which is responsible for focusing central vision of the eye and controls. AMD is diagnosed and being either dry (non-neovascular) or wet (neovascular) AMD. With dry AMD, the tissue of the macula gradually becomes thin and stops working properly. About 90% of patients with AMD are diagnosed as having the dry form. Wet AMD occurs when fluids leak from newly formed blood vessels under the macula. Although wet AMD represents a smaller diagnosed population of AMD, the wet form poses a much greater threat as vision loss can be much more rapid and severe.

Tyrosine kinase inhibitors (TKIs) have recently shown promise for treating AMD. See e.g., JAMA Ophthalmol, 2017 July 1; 135(7):676-768 and Exp Eye Res. 2018 March; 168:2-11. The problem with TKIs, however, is that systemic administration is not ideal because high levels of systemic dosing is required to achieve effective intraocular concentrations. This leads to the increased incidence of unacceptable side effects. Similarly, ocular instillation of TKIs and other drugs is most of the time ineffective because therapeutic levels of drug in the middle or back portions of the eye are often not achieved and drug concentration is difficult to control due to wash out, user error, and other factors. Other local therapy routes such as intravitreal injection have failed because such delivery routes tend to result in short half-life and rapid clearance, without sustained release capability being attained. Additionally, daily injections are frequently required to maintain therapeutic ocular drug levels, which is not tolerable to many patients. Some TKIs, such as axitinib, are poorly soluble and are injected as a suspension. These solid particulates, however, can aggregate, migrate or settle onto the retina and lead to local contact toxicity, holes, and floaters.

The use of ocular implants for drug delivery offers many advantages over traditional drops or injections. These implants are typically placed in or adjacent to the target eye tissues and offer better drug release and treatment duration potential. Although ocular implant devices have improved over the years, there are still many deficiencies. First, not all ocular implants are biodegradable. The need for chronic therapy can lead to accumulation of empty implants or may require tedious removal procedures following drug administration. Additionally, most biodegradable implants do not completely dissolve or do not dissolve in a time coinciding to drug release. The user is therefore left with implant vehicle residues commonly called floaters. Next, most ocular implants consist of complicated multiple layers requiring extensive manufacturing processes. This leads to increased production costs and time, and raises the likelihood of contamination from additional handling. Also, formulations containing hydrophobic drugs with biodegradable matrices can result in high initial drug burst or very little or no release of drug until erosion of the network occurs. This can lead to drug-dumping, which provides little benefit and causes toxicity issues.

### SUMMARY

Provided herein are biodegradable ocular hydrogel implants comprising a tyrosine kinase inhibitor (TKI) and a polymer network, and their use for the treatment of ocular conditions.

The disclosed hydrogel implants were effective in delivering efficacious doses of solubilized axitinib to the posterior segment of the eye, resulting in the successful treatment of vascular endothelial growth factor (VEGF)-induced retinal leakage for a duration of at least six months. See e.g., the exemplification section below where leakage scores in eyes treated with hydrogel implant show minimal to no vascular leakage through 12 months of TKI delivery.

In one aspect, the disclosed hydrogel implants are designed to comprise a clearance zone on the implant surface that is devoid of particulate TKI (e.g., undissolved TKI particles) prior to drug release. In one aspect, the the TKI is present in the hydrogel at or near saturation level, provided the TKI is not present in the clearance zone. In one aspect, this clearance zone provides a barrier between the TKI comprised in the hydrogel and the retinal cells of the eye, and prevents non-solubilized drug matter from releasing into the eye.

In one aspect, the TKI is present in the hydrogel at or near saturation level in the clearance zone. As drug release occurs, particulate TKI is solubilized, passes through the clearance zone and releases into the eye.

In one aspect, the rate of TKI release from the hydrogel is controlled by the drug solubility in the hydrogel matrix, which is determined by the chemical properties (e.g., molecular weight, solubility, crystallinity, etc.) and structure (e.g., multi-armed, crosslinked, branched, etc.) of the polymer network, as well as the overall surface area of the hydrogel.

In one aspect, the disclosed hydrogel implants are designed to be fully biodegradable, e.g., residual particulate matter or floaters are not of concern.

In one aspect, substantially all TKI is released from the hydrogel before degradation of the hydrogel occurs.

### BRIEF DESCRIPTION OF THE FIGURES

FIG. 1 illustrates the dispersion of TKI and outer clearance zone of one aspect of the disclosed hydrogel.

FIG. 2 shows the results from a PK/PD challenge of an inventive hydrogel implant over a 12 month duration.

### DETAILED DESCRIPTION

Provided herein are sustained-release biodegradable ocular hydrogel implants comprising a tyrosine kinase inhibitor (TKI), a polymer network, and a clearance zone.

### 1. Definitions

The term “biodegradable” refers to a material, such as the disclosed hydrogel implants, which degrades in vivo. Degradation of the material occurs over time and may occur concurrently with, or subsequent to, release of the TKI. In one aspect, “biodegradable” means that complete dissolution of the implant occurs, i.e., there is no residual hydrogel implant matter in the eye. In an alternative aspect, degradation may occur independently of TKI release such that e.g., residual TKI remains following degradation.

The term “polymer network” refers to a group of polymers comprising multiple branch structures (also referred to as “arms”) cross-linked to other polymer chains. The polymer chains may be of the same or different chemical structures, e.g., as in complementary or non-complementary repeating units.

Nomenclature for synthetic precursors used to generate the disclosed polymer networks are referenced using the number of arms followed by the MW of the PEG and then the reactive group (e.g., electrophile or nucleophile). For example 4a20K PEG SAZ refers to a 20,000 Da PEG with 4 arms with a succinimidylazelate end group, 4a20K PEG SAP refers to a 20,000 Da PEG with 4 arms with a succinimidyladipate end group, 4a20K PEG SG refers to a 20,000 Da PEG with 4 arms with a succinimidylglutarate end group, 4a20K PEG SS refers to a 20,000 Da PEG with 4 arms with a succinimidylsuccinate end group, etc. Similarly, 4a20K PEG NH2 means a 20,000 Da PEG with 4 arms with an amine end group, 8a20K PEG NH2 means a 20,000 Da PEG with 8 arms with an amine end group, etc.

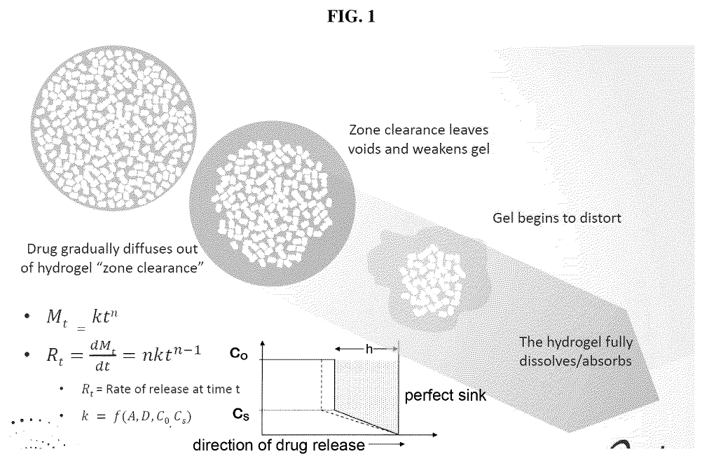

As used herein, “clearance zone” refers to a portion of the implant composed of hydrogel which is devoid of undissolved TKI particles prior to, or following the release of the TKI. “Clearance zone” and “zone clearance” are used interchangeably. An exemplary representation of the clearance zone is depicted in FIG. 1. As shown, the clearance zone provides a protective barrier between the TKI comprised in the hydrogel and the retinal cells in the eye. Without being bound by theory, this is because the poor water solubility of the TKI prevents high TKI concentrations on the outer surface of the implant which may contact tissue. As the properties of the polymer network change, e.g., as the polymer network slowly degrades, TKI continues to be released from the hydrogel by first passing through the clearance zone before it is released and comes in direct contact with the eye. In one aspect, the release of the TKI is solubility driven and is not affected by polymer network changes, except for dimensional changes that accompany polymer changes. In some aspects, the overall size of the clearance zone increases as more TKI is released from the hydrogel. In one aspect, there is a desire to match the size of the clearance zone and the rate of degradation of the hydrogel. For example, the polymer hydrolysis rate is matched to the TKI solubility so that as the size of the clearance zone increases, the hydrogel degradation increases so that hydrogel disappearance coincides roughly with TKI disappearance.

The term “amorphous” refers to a polymer or polymer network which does not exhibit crystalline structures in X-ray or electron scattering experiments.

The term “semi-crystalline” refers to a polymer or polymer network which possesses some crystalline character, i.e., exhibits crystalline properties in thermal analysis, X-ray scattering or electron scattering experiments. In some aspects, “semi-crystalline” polymers or networks of polymers have a highly ordered molecular structure with sharp melt points. In some aspects, “semi-crystalline” polymers or networks of polymers do not gradually soften with a temperature increase and instead remain solid until a given quantity of heat is absorbed and then rapidly change into a rubber or liquid.

As used herein, “homogenously dispersed” means the component, such as the TKI, is uniformly dispersed throughout the hydrogel or polymer network, except for the portion comprising the clearance zone.

The term “treat”, “treating”, or “treatment” refer to reversing, alleviating, delaying the onset of, or inhibiting the progress of an ocular condition, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “ocular condition” refers to a disease, ailment, or condition in which the eye, a region of the eye, or part of the eye is affected.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

### 2. Sustained-Release Biodegradable Ocular Hydrogel Implants

In a first embodiment, the clearance zone of the disclosed hydrogel implants are devoid of undissolved TKI particles prior to TKI release. By way of example, in one aspect of this embodiment, the particulate TKI is comprised in the polymer network of the hydrogel, but is not present in the clearance zone. In one aspect, based on the design and properties of the polymer network, only the dissolved TKI passes through the clearance zone and out of the hydrogel and into the eye.

In a second embodiment, the particulate TKI described herein is not in contact with retinal cells when the particulate TKI is comprised inside the hydrogel implant. Remaining features of the hydrogel implant are described herein e.g., as in the first embodiment.

In a third embodiment, the TKI described herein is dissolved prior to release into the eye, wherein the remaining features of the hydrogel implant are described herein e.g., as in the first or second embodiment.

In a fourth embodiment, the TKI described herein becomes solubilized before it enters or passes through the clearance zone, wherein the remaining features of the hydrogel implant are described herein e.g., as in the first, second, or third embodiment.

In a fifth embodiment, the TKI described herein is dissolved as it enters or passes through the clearance zone, wherein the remaining features of the hydrogel implant are described herein e.g., as in the first, second, third, or fourth embodiment.

In a sixth embodiment, the dissolved TKI described herein is present in the hydrogel implant at or near its saturation level, wherein the remaining features of the hydrogel implant are described herein e.g., as in the first, second, third, fourth or fifth embodiment.

In a seventh embodiment, the size of the clearance zone increases as a function of the amount of TKI release, wherein the remaining features of the hydrogel implant are described herein e.g., as in the first, second, third, fourth, fifth, or sixth embodiment.

In an eighth embodiment, the hydrogel implant is fully degraded following complete release of the TKI, wherein the remaining features of the hydrogel implant are described herein e.g., as in the first, second, third, fourth, fifth, sixth, or seventh embodiment. Alternatively, as part of an eighth embodiment, the hydrogel implant is fully degraded after about 12 months, after about 11 months, after about 10 months, after about 9 months, after about 8 months, after about 6 months, after about 5 months, after about 4 months, after about 3 months, after about 2 months, after about 1 month (i.e., after about 30 days) following complete release of the TKI, wherein the remaining features of the hydrogel implant are described herein e.g., as in the first, second, third, fourth, fifth, sixth, or seventh embodiment. Alternatively, as part of an eighth embodiment, the hydrogel implant is fully degraded following at least 80%, at least 85%, or at least 90% (e.g., at least 91%, at least 92%, at least 93%, at least 94%, at least 95%, at least 96%, at least 97%, at least 98%, or at least 99%) release of the TKI, wherein the remaining features of the hydrogel implant are described herein e.g., as in the first, second, third, fourth, fifth, sixth, or seventh embodiment.

In a ninth embodiment, the polymer network of the disclosed hydrogel implants comprises a plurality of polyethylene glycol (PEG) units, wherein the remaining features of the hydrogel are described herein e.g., as in the first, second, third, fourth, fifth, sixth, seventh, or eighth embodiment.

In a tenth embodiment, the polymer network of the disclosed hydrogel implants comprises a plurality of multi-arm PEG units, wherein the remaining features of the hydrogel are described herein e.g., as in the first, second, third, fourth, fifth, sixth, seventh, eighth, or ninth embodiment.

In an eleventh embodiment, the polymer network of the disclosed hydrogel implants comprises a plurality of multi-arm PEG units having at least 2 arms, wherein the remaining features of the hydrogel are described herein e.g., as in the first, second, third, fourth, fifth, sixth, seventh, eighth, ninth, or tenth embodiment. Alternatively, as part of an eleventh embodiment, the polymer network of the disclosed hydrogel implants comprises a plurality of multi-arm PEG units having from 2 to 10 arms, wherein the remaining features of the hydrogel are described herein e.g., as in the first, second, third, fourth, fifth, sixth, seventh, eighth, ninth, or tenth embodiment. In another alternative, as part of an eleventh embodiment, the polymer network of the disclosed hydrogel implants comprises a plurality of multi-arm PEG units having from 4 to 8 arms, wherein the remaining features of the hydrogel are described herein e.g., as in the first, second, third, fourth, fifth, sixth, seventh, eighth, ninth, or tenth embodiment. In another alternative, as part of an eleventh embodiment, the polymer network of the disclosed hydrogel implants comprises a plurality of 4-arm PEG units, wherein the remaining features of the hydrogel are described herein e.g., as in the first, second, third, fourth, fifth, sixth, seventh, eighth, ninth, or tenth embodiment. In another alternative, as part of an eleventh embodiment, the polymer network of the disclosed hydrogel implants comprises a plurality of 8-arm PEG units, wherein the remaining features of the hydrogel are described herein e.g., as in the first, second, third, fourth, fifth, sixth, seventh, eighth, ninth, or tenth embodiment.

In a twelfth embodiment, the polymer network of the disclosed hydrogel implants comprises a plurality of PEG units having a number average molecular weight (Mn) of at least 10,000 daltons, wherein the remaining features of the hydrogel are described herein e.g., as in the first, second, third, fourth, fifth, sixth, seventh, eighth, ninth, tenth, or eleventh embodiment. Alternatively, as part of a twelfth embodiment, the polymer network of the disclosed hydrogel implants comprises a plurality of PEG units having an Mn of at least 15,000 daltons, wherein the remaining features of the hydrogel are described herein e.g., as in the first, second, third, fourth, fifth, sixth, seventh, eighth, ninth, tenth, or eleventh embodiment. In another alternative, as part of a twelfth embodiment, the polymer network of the disclosed hydrogel implants comprises a plurality of PEG units having an Mn of at least 20,000 daltons, wherein the remaining features of the hydrogel are described herein e.g., as in the first, second, third, fourth, fifth, sixth, seventh, eighth, ninth, tenth, or eleventh embodiment. In another alternative, as part of a twelfth embodiment, the polymer network of the disclosed hydrogel implants comprises a plurality of PEG units having an Mn of at least 40,000 daltons, wherein the remaining features of the hydrogel are described herein e.g., as in the first, second, third, fourth, fifth, sixth, seventh, eighth, ninth, tenth, or eleventh embodiment. In another alternative, as part of a twelfth embodiment, the polymer network of the disclosed hydrogel implants comprises a plurality of PEG units having an Mn ranging from 10,000 daltons to 50,000 daltons, wherein the remaining features of the hydrogel are described herein e.g., as in the first, second, third, fourth, fifth, sixth, seventh, eighth, ninth, tenth, or eleventh embodiment. In another alternative, as part of a twelfth embodiment, the polymer network of the disclosed hydrogel implants comprises a plurality of PEG units having an Mn ranging from 10,000 daltons to 40,000 daltons, wherein the remaining features of the hydrogel are described herein e.g., as in the first, second, third, fourth, fifth, sixth, seventh, eighth, ninth, tenth, or eleventh embodiment. In another alternative, as part of a twelfth embodiment, the polymer network of the disclosed hydrogel implants comprises a plurality of PEG units having an Mn ranging from 30,000 daltons to 50,000 daltons, wherein the remaining features of the hydrogel are described herein e.g., as in the first, second, third, fourth, fifth, sixth, seventh, eighth, ninth, tenth, or eleventh embodiment. In another alternative, as part of a twelfth embodiment, the polymer network of the disclosed hydrogel implants comprises a plurality of PEG units having an Mn ranging from 35,000 daltons to 45,000 daltons, wherein the remaining features of the hydrogel are described herein e.g., as in the first, second, third, fourth, fifth, sixth, seventh, eighth, ninth, tenth, or eleventh embodiment. In another alternative, as part of a twelfth embodiment, the polymer network of the disclosed hydrogel implants comprises a plurality of PEG units having an Mn ranging from 15,000 daltons to 30,000 daltons, wherein the remaining features of the hydrogel are described herein e.g., as in the first, second, third, fourth, fifth, sixth, seventh, eighth, ninth, tenth, or eleventh embodiment. In another alternative, as part of a twelfth embodiment, the polymer network of the disclosed hydrogel implants comprises a plurality of PEG units having an Mn ranging from 15,000 daltons to 25,000 daltons, wherein the remaining features of the hydrogel are described herein e.g., as in the first, second, third, fourth, fifth, sixth, seventh, eighth, ninth, tenth, or eleventh embodiment. In another alternative, as part of a twelfth embodiment, the polymer network of the disclosed hydrogel implants comprises a plurality of PEG units having an Mn of about 20,000 daltons, wherein the remaining features of the hydrogel are described herein e.g., as in the first, second, third, fourth, fifth, sixth, seventh, eighth, ninth, tenth, or eleventh embodiment. In another alternative, as part of a twelfth embodiment, the polymer network of the disclosed hydrogel implants comprises a plurality of PEG units having an Mn of about 40,000 daltons, wherein the remaining features of the hydrogel are described herein e.g., as in the first, second, third, fourth, fifth, sixth, seventh, eighth, ninth, tenth, or eleventh embodiment.

In a thirteenth embodiment, the polymer network of the disclosed hydrogel implants comprises a plurality of PEG units crosslinked by a hydrolyzable linker, wherein the remaining features of the hydrogel are described herein e.g., as in the first, second, third, fourth, fifth, sixth, seventh, eighth, ninth, tenth, or eleventh, or twelfth embodiment. Alternatively, as part of a thirteenth embodiment, the polymer network of the disclosed hydrogel implants comprises a plurality of PEG units crosslinked by a hydrolyzable linker having the formula:

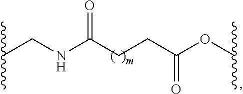

wherein m is an integer from 1 to 9, wherein the remaining features of the hydrogel are described herein e.g., as in the first, second, third, fourth, fifth, sixth, seventh, eighth, ninth, tenth, or eleventh, or twelfth embodiment. In another alternative, the polymer network of the disclosed hydrogel implants comprises a plurality of PEG units crosslinked by a hydrolyzable linker having the formula:

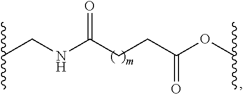

wherein m is an integer from 2 to 6, wherein the remaining features of the hydrogel are described herein e.g., as in the first, second, third, fourth, fifth, sixth, seventh, eighth, ninth, tenth, or eleventh, or twelfth embodiment. In another alternative, as part of a thirteenth embodiment, the polymer network of the disclosed hydrogel implants comprises a plurality of PEG units having the formula:

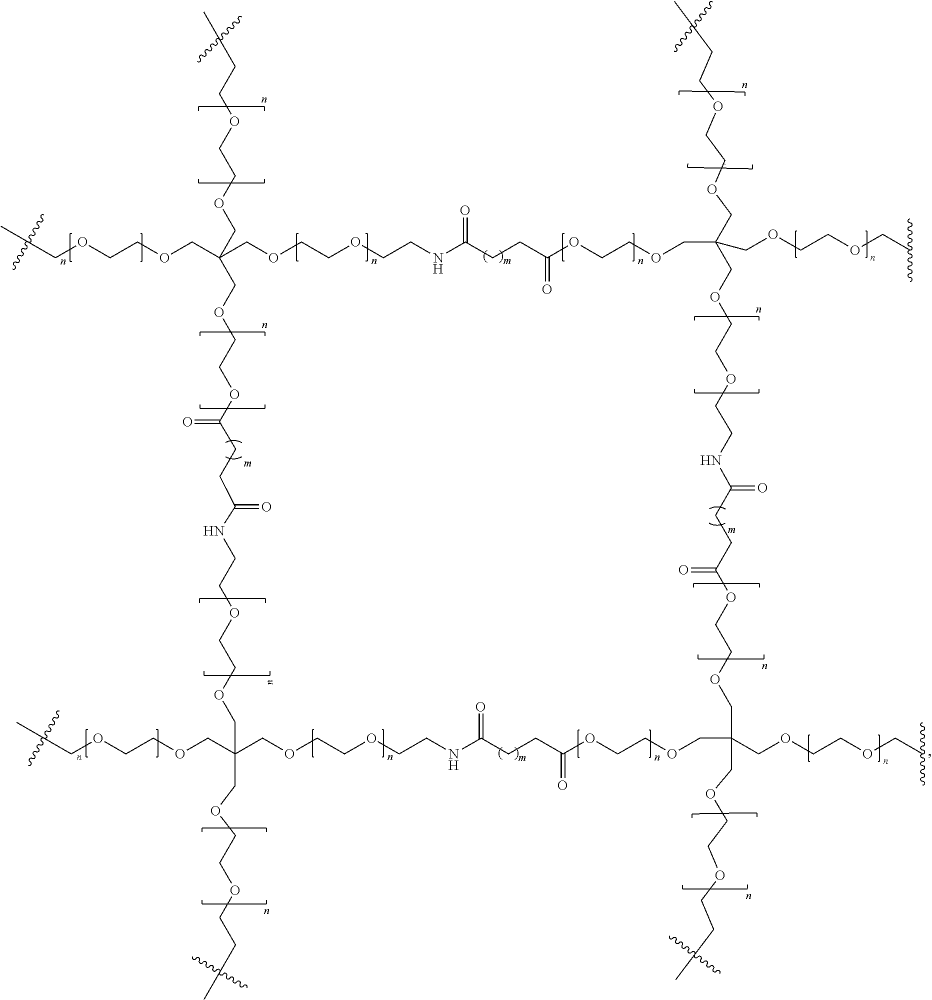

wherein n represents an ethylene oxide repeating unit and the wavy lines represent the points of repeating units of the polymer network, wherein the remaining features of the hydrogel are described herein e.g., as in the first, second, third, fourth, fifth, sixth, seventh, eighth, ninth, tenth, or eleventh, or twelfth embodiment. In another alternative, as part of a thirteenth embodiment, the polymer network of the disclosed compositions comprise a plurality of PEG units having the formula set forth above, but with an 8-arm PEG scaffold.

In a fourteenth embodiment, the polymer network of the disclosed hydrogel implant is formed by reacting a plurality of polyethylene glycol (PEG) units comprising groups which are susceptible to nucleophilic attack with one or more nucleophilic groups to form the polymer network, wherein the remaining features of the hydrogel are described herein e.g., as in the first, second, third, fourth, fifth, sixth, seventh, eighth, ninth, tenth, or eleventh, or twelfth embodiment. Examples of suitable groups which are susceptible to nucleophilic attack include, but art not limited to activated esters (e.g., thioesters, succinimidyl esters, benzotriazolyl esters, esters of acrylic acids, and the like). Examples of suitable nucleophilic groups include, but art not limited to, amines and thiols.

In a fifteenth embodiment, the polymer network of the disclosed hydrogel implant is formed by reacting a plurality of polyethylene glycol (PEG) units, each having a molecule weight as described above in the twelfth embodiment and which comprise groups which are susceptible to nucleophilic attack, with one or more nucleophilic groups to form the polymer network, wherein the remaining features of the hydrogel are described herein e.g., as in the first, second, third, fourth, fifth, sixth, seventh, eighth, ninth, tenth, or eleventh, or fourteenth embodiment. Alternatively, as part of a fifteenth embodiment, the polymer network of the disclosed hydrogel implant is formed by reacting a plurality of polyethylene glycol (PEG) units, each having a molecule weight as described above in the twelfth embodiment and which comprise a succinimidyl ester group, with one or more nucleophilic groups to form the polymer network, wherein the remaining features of the hydrogel are described herein e.g., as in the first, second, third, fourth, fifth, sixth, seventh, eighth, ninth, tenth, or eleventh, or fourteenth embodiment. In another alternative, as part of a fifteenth embodiment, the polymer network of the disclosed hydrogel implant is formed by reacting a plurality of polyethylene glycol (PEG) units selected from 4a20K PEG SAZ, 4a20K PEG SAP, 4a20K PEG SG, 4a20K PEG SS, 8a20K PEG SAZ, 8a20K PEG SAP, 8a20K PEG SG, and 8a20K PEG SS, wherein the remaining features of the hydrogel are described herein e.g., as in the first, second, third, fourth, fifth, sixth, seventh, eighth, ninth, tenth, or eleventh, twelfth, thirteenth, or fourteenth embodiment.

In a sixteenth embodiment, the polymer network of the disclosed hydrogel implant is formed by reacting a plurality of polyethylene glycol (PEG) units comprising groups which are susceptible to nucleophilic attack with one or more amine groups to form the polymer network, wherein the remaining features of the hydrogel are described herein e.g., as in the first, second, third, fourth, fifth, sixth, seventh, eighth, ninth, tenth, or eleventh, twelfth, fourteenth, or fifteenth embodiment. Alternatively, as part of a sixteenth embodiment, the polymer network of the disclosed hydrogel implant is formed by reacting a plurality of polyethylene glycol (PEG) units comprising groups which are susceptible to nucleophilic attack with one or more PEG or Lysine based-amine groups to form the polymer network, wherein the remaining features of the hydrogel are described herein e.g., as in the first, second, third, fourth, fifth, sixth, seventh, eighth, ninth, tenth, or eleventh, twelfth, fourteenth, or fifteenth embodiment. In another alternative, as part of a sixteenth embodiment, the polymer network of the disclosed hydrogel implant is formed by reacting a plurality of polyethylene glycol (PEG) units comprising groups which are susceptible to nucleophilic attack with one or more PEG or Lysine based-amine groups selected from 4a20K PEG NH2, 8a20K PEG NH2, and trilysine, or salts thereof, wherein the remaining features of the hydrogel are described herein e.g., as in the first, second, third, fourth, fifth, sixth, seventh, eighth, ninth, tenth, or eleventh, twelfth, fourteenth, or fifteenth embodiment. In another alternative, as part of a sixteenth embodiment, the polymer network of the disclosed hydrogel implant is formed by reacting 4a20K PEG-SAZ with 8a20K PEG NH2, wherein the remaining features of the hydrogel are described herein e.g., as in the first, second, third, fourth, fifth, sixth, seventh, eighth, ninth, tenth, or eleventh, twelfth, fourteenth, or fifteenth embodiment. In another alternative, as part of a sixteenth embodiment, the polymer network of the disclosed hydrogel implant is formed by reacting 2 parts 4a20K PEG-SAZ with 1 part 8a20K PEG NH2, wherein the remaining features of the hydrogel are described herein e.g., as in the first, second, third, fourth, fifth, sixth, seventh, eighth, ninth, tenth, or eleventh, twelfth, fourteenth, or fifteenth embodiment.

In a seventeenth embodiment, the ocular hydrogel implant is amorphous (e.g., under aqueous conditions such as in vivo), wherein the remaining features of the hydrogel are described herein e.g., as in the first, second, third, fourth, fifth, sixth, seventh, eighth, ninth, tenth, or eleventh, twelfth, thirteenth, fourteenth, fifteenth, or sixteenth embodiment.

In an eighteenth embodiment, the ocular hydrogel implant is semi-crystalline (e.g., in the absence of water), wherein the remaining features of the hydrogel are described herein e.g., as in the first, second, third, fourth, fifth, sixth, seventh, eighth, ninth, tenth, or eleventh, twelfth, thirteenth, fourteenth, fifteenth, sixteenth, or seventeenth embodiment.

In a nineteenth embodiment, the tyrosine kinase inhibitor of the disclosed hydrogel implant is homogenously dispersed as a particulate within the polymer network, wherein the remaining features of the hydrogel are described herein e.g., as in the first, second, third, fourth, fifth, sixth, seventeenth, or eighteenth embodiment.

In a twentieth embodiment, the tyrosine kinase inhibitor of the hydrogel is released over a period of at least 15 days, wherein the remaining features of the hydrogel are described herein e.g., as in the first, second, third, fourth, fifth, sixth, seventeenth, eighteenth, or nineteenth embodiment. Alternatively, as part of a twentieth embodiment, the tyrosine kinase inhibitor of the disclosed hydrogel implant is released over a period of at least 30 days, wherein the remaining features of the hydrogel are described herein e.g., as in the first, second, third, fourth, fifth, sixth, seventeenth, eighteenth, or nineteenth embodiment. In another alternative, as part of a twentieth embodiment, the tyrosine kinase inhibitor of the disclosed hydrogel implant is released over a period of at least 60 days, wherein the remaining features of the hydrogel are described herein e.g., as in the first, second, third, fourth, fifth, sixth, seventeenth, eighteenth, or nineteenth embodiment. In another alternative, as part of a twentieth embodiment, the tyrosine kinase inhibitor of the disclosed hydrogel implant is released over a period of at least 90 days, wherein the remaining features of the hydrogel are described herein e.g., as in the first, second, third, fourth, fifth, sixth, seventeenth, eighteenth, or nineteenth embodiment. In another alternative, as part of a twentieth embodiment, the tyrosine kinase inhibitor of the disclosed hydrogel implant is released over a period of at least 120 days, wherein the remaining features of the hydrogel are described herein e.g., as in the first, second, third, fourth, fifth, sixth, seventeenth, eighteenth, or nineteenth embodiment. In another alternative, as part of a twentieth embodiment, the tyrosine kinase inhibitor of the disclosed hydrogel implant is released over a period of at least 150 days, wherein the remaining features of the hydrogel are described herein e.g., as in the first, second, third, fourth, fifth, sixth, seventeenth, eighteenth, or nineteenth embodiment. In another alternative, as part of a twentieth embodiment, the tyrosine kinase inhibitor of the disclosed hydrogel implant is released over a period of at least 180 days, wherein the remaining features of the hydrogel are described herein e.g., as in the first, second, third, fourth, fifth, sixth, seventeenth, eighteenth, or nineteenth embodiment. In another alternative, as part of a twentieth embodiment, the tyrosine kinase inhibitor of the disclosed hydrogel implant is released over a period of at least 210 days, wherein the remaining features of the hydrogel are described herein e.g., as in the first, second, third, fourth, fifth, sixth, seventeenth, eighteenth, or nineteenth embodiment. In another alternative, as part of a twentieth embodiment, the tyrosine kinase inhibitor of the disclosed hydrogel implant is released over a period of at least 240 days, wherein the remaining features of the hydrogel are described herein e.g., as in the first, second, third, fourth, fifth, sixth, seventeenth, eighteenth, or nineteenth embodiment. In another alternative, as part of a twentieth embodiment, the tyrosine kinase inhibitor of the disclosed hydrogel implant is released over a period of at least 270 days, wherein the remaining features of the hydrogel are described herein e.g., as in the first, second, third, fourth, fifth, sixth, seventeenth, eighteenth, or nineteenth embodiment. In another alternative, as part of a twentieth embodiment, the tyrosine kinase inhibitor of the disclosed hydrogel implant is released over a period of at least 300 days, wherein the remaining features of the hydrogel are described herein e.g., as in the first, second, third, fourth, fifth, sixth, seventeenth, eighteenth, or nineteenth embodiment. In another alternative, as part of a twentieth embodiment, the tyrosine kinase inhibitor of the disclosed hydrogel implant is released over a period of at least 330 days, wherein the remaining features of the hydrogel are described herein e.g., as in the first, second, third, fourth, fifth, sixth, seventeenth, eighteenth, or nineteenth embodiment. In another alternative, as part of a twentieth embodiment, the tyrosine kinase inhibitor of the disclosed hydrogel implant is released over a period of at least 365 days, wherein the remaining features of the hydrogel are described herein e.g., as in the first, second, third, fourth, fifth, sixth, seventeenth, eighteenth, or nineteenth embodiment.

In a twenty-first embodiment, the tyrosine kinase inhibitor of the disclosed hydrogel implant is in the form of an encapsulated microparticle, wherein the remaining features of the hydrogel are described herein e.g., as in the first, second, third, fourth, fifth, sixth, seventeenth, eighteenth, nineteenth, or twentieth embodiment.

In a twenty-second embodiment, the tyrosine kinase inhibitor of the disclosed hydrogel implant has an appropriate aqueous solubility for the desired release rate, wherein the remaining features of the hydrogel are described herein e.g., as in the first, second, third, fourth, fifth, sixth, seventeenth, eighteenth, nineteenth, twentieth, or twenty-first embodiment.

In a twenty-third embodiment, the tyrosine kinase inhibitor of the disclosed hydrogel implant is in the form of an encapsulated microparticle comprising poly (lactic-co-glycolic acid, wherein the remaining features of the hydrogel are described herein e.g., as in the first, second, third, fourth, fifth, sixth, seventeenth, eighteenth, nineteenth, twentieth, twenty-first, or twenty-second embodiment.

In a twenty-fourth embodiment, the tyrosine kinase inhibitor of the disclosed hydrogel implant is selected from abemaciclib, acalabrutinib, afatinib, alectinib, axitinib, barictinib, binimetinib, brigatinib, cabozantinib, ceritinib, coblmetinib, crizotinib, dabrafenib, dacomitinib, dasatinib, encorafenib, erlotinib, everolimus, fostamatinib, gefitinib, gilteritinib, ibrutinib, imatinib, larotrectinib, lenvatinib, lorlatinib, axitinib, idelalisib, lenvatinib, midostaurin, neratinib, netarsudil, nilotinib, nintedanib, osimertinib, palbociclib, pazopanib, ponatinib, regorafenib, ribociclib, ruxolitinib, sirolimus, sorafenib, sunitinib, temsirolimus, tofacitinib, trametinib, vandetanib, and vemurafenib, wherein the remaining features of the hydrogel are described herein e.g., as in the first, second, third, fourth, fifth, sixth, seventeenth, eighteenth, nineteenth, twentieth, twenty-first, twenty-second, or twenty-third embodiment. Alternatively, the tyrosine kinase inhibitor of the hydrogel is sunitinib, nintedanib, regorefanib, or axitinib, wherein the remaining features of the hydrogel are described herein e.g., as in the first, second, third, fourth, fifth, sixth, seventeenth, eighteenth, nineteenth, twentieth, twenty-first, twenty-second, or twenty-third embodiment. In another alternative, the tyrosine kinase inhibitor of the hydrogel is axitinib, wherein the remaining features of the hydrogel are described herein e.g., as in the first, second, third, fourth, fifth, sixth, seventeenth, eighteenth, nineteenth, twentieth, twenty-first, twenty-second, or twenty-third embodiment. In another alternative, the tyrosine kinase inhibitor of the hydrogel is one that targets VEGFR1, wherein the remaining features of the hydrogel are described herein e.g., as in the first, second, third, fourth, fifth, sixth, seventeenth, eighteenth, nineteenth, twentieth, twenty-first, twenty-second, or twenty-third embodiment. In another alternative, the tyrosine kinase inhibitor of the hydrogel is one that targets VEGFR2, wherein the remaining features of the hydrogel are described herein e.g., as in the first, second, third, fourth, fifth, sixth, seventeenth, eighteenth, nineteenth, twentieth, twenty-first, twenty-second, or twenty-third embodiment.

In a twenty-fifth embodiment, the ocular hydrogel described herein is formulated as intravitereal implant that can be delivered to the eye e.g., via a needle injection, wherein the remaining features of the hydrogel are described herein e.g., as in the first, second, third, fourth, fifth, sixth, seventeenth, eighteenth, nineteenth, twentieth, twenty-first, twenty-second, twenty-third, or twenty-fourth embodiment.

In a twenty-sixth embodiment, the ocular hydrogel described herein is affixed to the lower punctum of the eye, wherein the remaining features of the hydrogel are described herein e.g., as in the first, second, third, fourth, fifth, sixth, seventeenth, eighteenth, nineteenth, twentieth, twenty-first, twenty-second, twenty-third, twenty-fourth, or twenty-fifth embodiment.

In a twenty-seventh embodiment, the ocular hydrogel described herein is injected into the vitreous humor, injected into the anterior chamber, or is affixed to the upper or lower punctum of the eye, wherein the remaining features of the hydrogel are described herein e.g., as in the first, second, third, fourth, fifth, sixth, seventeenth, eighteenth, nineteenth, twentieth, twenty-first, twenty-second, twenty-third, twenty-fourth, or twenty-fifth embodiment.

### 2. Methods, Processes, and Use

The disclosed hydrogel implants are useful in treating ocular conditions. Thus, provided herein are methods of treating one or more ocular conditions described herein, comprising affixing the disclosed hydrogel implants to the eye of a subject e.g., to the lower punctum of the eye. Also provided is the use of the disclosed hydrogel implants in the manufacture of medicaments for treating one or more ocular conditions described herein.

In one aspect, the disclosed hydrogel implants are useful in treating ocular conditions associated with maculopathies/rentinal degeneration. Such conditions include e.g., Age Related Macular Degeneration (AMD) such as wet or dry AMD, Choroidal Neovascularization, Diabetic Retinopathy, Acute Macular Neuroretinopathy, Central Serous Chorioretinopathy, Cystoid Macular Edema, and Diabetic Macular Edema.

In one aspect, the disclosed hydrogel implants are useful in treating ocular conditions associated with uveitis/retinitis/choroiditis. Such conditions include e.g., Acute Multifocal Placoid Pigment Epitheliopathy, Behcet's Disease, Birdshot Retinochoroidopathy, Infectious (Syphilis, Lyme, Tuberculosis, Toxoplasmosis), Intermediate Uveitis (Pars Planitis), Multifocal Choroiditis, Multiple Evanescent White Dot Syndrome (MEWDS), Ocular Sarcoidosis, Posterior Scleritis, Serpignous Choroiditis, Subretinal Fibrosis and Uveitis Syndrome, and Vogt-Koyanagi-Harada Syndrome.

In one aspect, the disclosed hydrogel implants are useful in treating ocular conditions associated with vascular diseases/exudative diseases. Such conditions include e.g., Coat's Disease, Parafoveal Telangiectasis, Papillophlebitis, Frosted Branch Angitis, Sickle Cell Retinopathy and other Hemoglobinopathies, Angioid Streaks, and Familial Exudative Vitreoretinopathy.

In one aspect, the disclosed hydrogel implants are useful in treating ocular conditions associated with trauma/surgery. Such conditions include e.g., Sympathetic Ophthalmia, Uveitic Retinal Disease, Retinal Detachment, Trauma, Photodynamic Laser Treatment, Photocoagulation, Hypoperfusion During Surgery, Radiation Retinopathy, and Bone Marrow Transplant Retinopathy.

In one aspect, the disclosed hydrogel implants are useful in treating ocular conditions associated with proliferative disorders. Such conditions include e.g., Proliferative Vitreal Retinopathy and Epiretinal Membranes, Proliferative Diabetic Retinopathy, and Retinopathy of Prematurity (retrolental fibroplastic).

In one aspect, the disclosed hydrogel implants are useful in treating ocular conditions associated with infectious disorders. Such conditions include e.g., Ocular Histoplasmosis, Ocular Toxocariasis, Presumed Ocular Histoplasmosis Syndrome (POHS), Endophthalmitis, Toxoplasmosis, Retinal Diseases Associated with HIV Infection, Choroidal Disease Associated with HIV Infection, Uveitic Disease Associated with HIV Infection, Viral Retinitis, Acute Retinal Necrosis, Progressive Outer Retinal Necrosis, Fungal Retinal Diseases, Ocular Syphilis, Ocular Tuberculosis, Diffuse Unilateral Subacute Neuroretinitis, and Myiasis.

In one aspect, the disclosed hydrogel implants are useful in treating ocular conditions associated with genetic disorders. Such conditions include e.g., Systemic Disorders with Associated Retinal Dystrophies, Congenital Stationary Night Blindness, Cone Dystrophies, Fundus Flavimaculatus, Best's Disease, Pattern Dystrophy of the Retinal Pigmented Epithelium, X-Linked Retinoschisis, Sorsby's Fundus Dystrophy, Benign Concentric Maculopathy, Bietti's Crystalline Dystrophy, pseudoxanthoma elasticum, Osler Weber syndrome.

In one aspect, the disclosed hydrogel implants are useful in treating ocular conditions associated with Retinal Tears/Holes. Such conditions include e.g., Detachment, Macular Hole, and Giant Retinal Tear.

In one aspect, the disclosed hydrogel implants are useful in treating ocular conditions associated with tumors. Such conditions include e.g., Retinal Disease Associated with Tumors, Solid Tumors, Tumor Metastasis, Benign Tumors, for example, hemangiomas, neurofibromas, trachomas, and pyogenic granulomas, Congenital Hypertrophy of the RPE, Posterior Uveal Melanoma, Choroidal Hemangioma, Choroidal Osteoma, Choroidal Metastasis, Combined Hamartoma of the Retina and Retinal Pigmented Epithelium, Retinoblastoma, Vasoproliferative Tumors of the Ocular Fundus, Retinal Astrocytoma, Intraocular Lymphoid Tumors.

In one aspect, the disclosed hydrogel implants are useful in treating Punctate Inner Choroidopathy, Acute Posterior Multifocal Placoid Pigment Epitheliopathy, Myopic Retinal Degeneration, Acute Retinal Pigment Epithelitis, Ocular inflammatory and immune disorders, ocular vascular malfunctions, Corneal Graft Rejection, and Neovascular Glaucoma.

Specific dosages and treatment regimens for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

Various techniques may be employed to produce the implants described herein. Useful techniques include, but are not necessarily limited to, solvent evaporation methods, phase separation methods, interfacial methods, molding methods, injection molding methods, extrusion methods, coextrusion methods, carver press method, die cutting methods, and the like.

### EXEMPLIFICATION

### Preparation of In Situ Gelling TKI Implant Formulations

A series of TKI formulations were prepared as described in detail in the table below.

| | | | | Buffer | | | Gel Time |
|---|---|---|---|---|---|---|---|
| | | API | Provisc | modifier | 4a20KSAZ | 8a20KNH3Cl | range |
| Ex. | TKI | (mg) | (mg) | (mg) | (mg) | (mg) | (min) |
| 1 | Sunitinib | 17.8 | 113.4 | 28.4 | 20.0 | 9.2 | 3.5-5 |
| 3 | Nintedanib | 24.0 | 154.4 | 38.6 | 20.0 | 9.2 | 3.5-5 |
| 4 | Regorefanib | 28.4 | 183.7 | 45.9 | 20.0 | 9.2 | 6-8 |
| 2 | Axitinib | 23.5 | 152.8 | 38.2 | 20.0 | 9.2 | 4.5-6 |
| 5 | Vehicle | 0.0 | 216.0 | 54.0 | 20.0 | 9.2 | 6-9 |

Provisc: Each mL of PROVISC (OVD) contains 10.0 mg sodium hyaluronate; 2.0 mg dibasic sodium phosphate, anhydrous; 0.45 mg monobasic sodium phosphate, monohydrate; 7.5 mg sodium chloride; hydrochloric acid and/or sodium hydroxide to adjust pH and water for injection. The sodium hyaluronateused in PROVISC (OVD) is derived from bacterial fermentation and has an average molecular weight of 2,500,000 Daltons. The osmolality of PROVISC (OVD) is 310 ± 50 mOsm/kg; the pH ranges from 6.8 to 7.5. Buffer modifier: 0.5 mg/ml Na Borate pH 6.8

### In Vivo Testing in in Situ Gelling TKI Implant Formulations

The samples prepared in examples 1-5 were tested in rabbit eyes. Briefly, on Day 0 rabbits were injected with 10 uL in the left and right eye (OU) with test articles as listed below. Animals were euthanized at the time points listed in the study design table below. Eyes were harvested, and fixed in Davidson's solution for histopathologic analysis.

A total of 10 left and right eyes from 5 rabbits were examined. A suture had been placed at the 12 o'clock position for orientation at harvest. Typically eyes were trimmed in half in the plane from 12 o'clock to 6 o'clock through the lens and optic nerve along the midline. This captures as many optic structures in one plane as is possible. The trimmed eyes were examined grossly and abnormalities noted. Each half of the globe trimmed was embedded in its own cassette. For each block 6 hematoxylin and eosin (H&E)-stained slides were prepared that were separated by 1000 microns (1 mm). Each slide contained 2 serial sections.

All slides were evaluated by a board-certified veterinary pathologist. Tissues were scored on a semi-quantitative scale from 0-5 for any abnormalities. See the table on page 5. The presence or absence of any injected material was also noted.

| Score | Description |
|---|---|
| 0 | No change; normal |
| 1 | Rare foci of change; minimal |
| 2 | Mild diffuse change or more pronounced focal change |
| 3 | Moderate diffuse change |
| 4 | Marked diffuse change |
| 5 | Severe diffuse change |

| | | | Mean Inflammation | Mean Retinal, | Mean Epithelial |
|---|---|---|---|---|---|
| | | | within the | Scleral, or | Hyperplasia |
| Example | | | Vitreous | Episcleral | in Front of |
| or Group | API | Eye | Chamber (0-5) | Inflammation (0-5) | Ora Serrata (0-5) |
| 1 | Sunitinib | OD | 0 | 0 | 0 |
| | | OS | .3 | 0 | 0 |
| 2 | Nintedanib | OD | 0 | 0 | 0 |
| | | OS | 0 | 0 | 0 |
| 3 | Regorefanib | OD | 0 | .1 | 0 |
| | | OS | .2 | .1 | 0 |
| 4 | Axitinib | OD | 0 | .1 | 0 |
| | | OS | 0 | 0 | 0 |
| 5 | Vehicle | OD | 0 | 0 | 0 |
| | | OS | 0 | 0 | 0 |

Under the conditions of the study intravitreal injection of rabbit eyes with formulations of hydrogel depots with tyrosine kinase inhibitors at 14 days post-injection resulted in the continued presence of the hydrogel in the vitreous chamber of at least one eye from each group except Group 1 and Group 3 where no hydrogel material was noted in either eye.

Inflammation was never present around any of the injected material observed in any of the eyes from Groups 2, 4, and 6. Minimal inflammation composed primarily of macrophages in the vitreous chamber and/or attached to the retina was observed in occasional samples from Groups 1 and 3, although not associated with injected material. Again, no injected material was observed in either eye from Group 1 or Group 3.

Minimal inflammation and fibrosis were observed in a few slide samples from Groups 3 and 4. These were typically small linear areas of fibrosis with a few macrophages admixed. They are interpreted as sequela to needle injection.

One or a few small areas of retinal disruption or retinal folds were observed in at least 1 eye from Groups 1, 3, 4 and 6. These could be retinal invaginations due to needle injection. A very small retinal detachment measuring 100 microns in length is present in one eye at the location of the small retinal disruption (Group 3). No other retinal detachments were noted in any eye from any group.

A focus of mild histiocytic and multi-nucleated inflammation was observed around a small displaced focus of lens fibers in the vitreous chamber of one eye from Group 3. This is considered lens-induced granulomatous endophthalmitis, and may be due to a slight nick of the lens by the needle at injection. No other such lesions were observed in any eye from any group.

### General Synthetic Methods

Inventive ocular hydrogel implants are prepared by the following procedure. This hydrogel implant comprises axitinib as the active pharmaceutical ingredient (API) and 4-arm polyethylene glycol (PEG) based hydrogel which serves as the inactive delivery platform.

| Step | Manufacturing Action |
|---|---|
| 1 | Cut polyurethane tubing in appropriate length pieces and insert |
| | dispensing tips into both ends of the tubing. |
| 2 | Formulate 8a20k free amine/sodium phosphate dibasic solution. |
| | Weigh solution into a syringe. |
| 3 | Weigh axitinib API into a syringe. |
| 4 | Mix axitinib API with 8a20k free amine/sodium phosphate |
| | solution. |
| 5 | Sonicate/vortex the combined axitinib/amine syringe. |
| 6 | Formulate 4a20k PEG SAZ/sodium phosphate monobasic solution. |
| | Weigh solution into a syringe. |
| 7 | Mix axitinib/amine syringe with PEG SAZ syringe. Cast the |
| | suspension through tubing, cap the dispensing tips to close the |
| | tubing, and allow the suspension to gel. Record the gel time. |
| | Repeat for the number of desired runs. |
| 8 | Allow the gelled formulation to cure. |
| 9 | Place the strands on a fixture. Dry the strands in an incubator |
| | with an N2 sweep. |
| 10 | Remove the dried strands and cut to the desired implant length. |

| Step | Manufacturing Action |
|---|---|
| 1 | Inspect each implant using a vision system for length, diameter, |
| | and visual appearance/foreign particulates. Discard any implants |
| | that do not meet specifications. |
| 2 | Load each passing implant into a needle. Place the protective cap |
| | on the needle. Transfer the loaded needles into a glove box. |
| 3 | Remove the protective caps and place loaded needles on the PEG- |
| | Tipping manifold. Dip into 1k PEG to tip the needles. |
| 4 | Place the manifold on the completed rack to allow the PEG Tip to |
| | anneal. |
| 5 | Place the protective cap on the needle, insert the needle hub into |
| | the luer lock end of the needle, and cap the luer lock end of the |
| | needle using a vented male luer cap. |
| 6 | Transfer foil pouches into the glove box. Place capped needles |
| | into the foil pouches. |
| 7 | Condition the foil pouches with the capped needles. |
| 8 | Seal the foil pouches and remove from glove box. |
| 9 | Transfer the sealed foil pouches in clearly labeled bags and |
| | store in a refrigerator prior to sterilization. |
| 10 | Complete required yield calculations. |
| 11 | Send the completed implants for gamma irradiation. |

### Clinical Investigation

### A. Reduction of Vascular Leakage in Rabbits

Micronized TKI particles were formulated in an inventive hydrogel matrix according to the procedure above. Thirty eyes of naïve Dutch belted rabbits (n=15) were bilaterally dosed with hydrogel implant at Day 0. Eyes were challenged with an injection of vascular endothelial growth factor (VEGF) at predetermined timepoints over the course of 12 months and were evaluated for leakage by fluorescein angiography (FA) and dilated fundus examination. At each VEGF challenge timepoint, eyes treated with inventive implant were compared to untreated control eyes (n=4). Ocular tissue was collected for pharmacokinetic (PK) analysis by liquid chromatography/mass spectrometry (LC/MS) immediately after FA evaluations up to 6 months. Tolerability was assessed via MacDonald Shadduck scores and clinical observations.

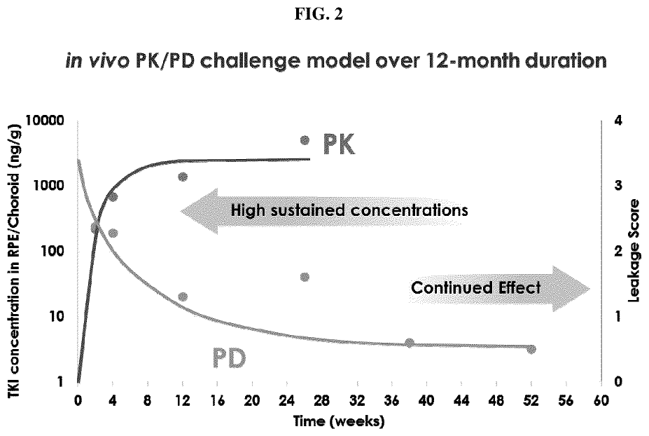

Inventive hydrogel implant treated eyes significantly suppressed leakage at 3, 6, 9 and 12 months when challenged with a VEGF suspension. The leakage scores in eyes treated with hydrogel implant show minimal to no vascular leakage through 12 months. Untreated control eyes showed high tortuosity and leakage at all timepoints. See FIG. 2. Retinal tissue showed high drug concentrations (>2800X IC50) through 6 months in the treated animals. MacDonald Shadduck scores showed the treatment was well-tolerated. Other clinical observations raised no safety concerns.

### B. Additional Clinical Studies Data in Rabbits

Inventive hydrogel implant test articles were formulated to contain approximately 110 μg of axitinib drug substance per implant. In biorelevant media (PBS, pH 7.2, 37° C.) the implants hydrated to a 24 hour dimension of approximately 0.5×9.2 mm.

A single hydrogel implant was administered to each eye (bilateral) of 14 female Dutch Belted rabbits via intravitreal injection for a total of 28 eyes. Two rabbits each were euthanized at study timepoints of days 1, 45, 90, 137, 180, 225 and 270. At euthanasia time points, plasma samples were taken and both eyes were enucleated and frozen in liquid nitrogen. The frozen eye tissues and plasma were stored at −80° C. until tested for bioanalysis. Samples were tested after the study midpoint (Day 137) and at the end of the study (Day 270). Four of 28 eyes were excluded from statistical analysis due to identified experimental errors.

Concentrations of axitinib in rabbit AH samples over the study duration (see Table 1) were considered low relative to the concentrations observed in the VH, retina and choroid indicating a low level of axitinib migration towards the anterior chamber from the posterior chamber. Median axitinib concentrations were maximal at 5.9 ng/ml at 45 days and declined to zero by 225 days.

TABLE 1
Summary of Axitinib Concentrations in Aqueous Humor ^{A}

| | | Average | Min | Median | Max | Std Dev | | 95% |
|---|---|---|---|---|---|---|---|---|
| Day | N | (ng/mL) | (ng/mL) | (ng/mL) | (ng/mL) | (ng/mL) | CV | CI |
| 1 | 4 | 0.5 | 0.0 | 0.5 | 0.9 | 0.4 | 75% | 0.4 |
| 45 | 3 | 2.7 | 0.8 | 1.5 | 5.9 | 2.8 | 102% | 3.1 |
| 90 | 3 | 1.0 | 0.4 | 0.5 | 2.0 | 0.9 | 89% | 1.0 |
| 137 | 4 | 0.7 | 0.6 | 0.7 | 1.0 | 0.2 | 23% | 0.2 |
| 180 | 4 | 0.2 | 0.0 | 0.2 | 0.6 | 0.3 | 112% | 0.2 |
| 225 | 3 | 0.0 | 0.0 | 0.0 | 0.0 | 0.0 | n.a. | n.a. |
| 270 | 3 | 0.0 | 0.0 | 0.0 | 0.1 | 0.1 | 173% | 0.1 |

n.a. denotes not applicable for statistical calculations.
^{A} The method for the determination of axitinib in AH had a LLOQ = 0.100 ng/mL

Median axitinib concentrations of soluble axitinib in rabbit VH samples over the study duration (see Table 2) were maximal (264.0 ng/ml) at 180 days. Individual samples ranged from a minimum of 2.9 ng/ml (days 225 and 270) to a maximum of 571.0 ng/ml (day 180).

TABLE 2
Summary of Soluble Axitinib Concentrations in Vitreous Humor ^{A}

| | | Average | Min | Median | Max | Std Dev | | 95% |
|---|---|---|---|---|---|---|---|---|
| Day | N | (ng/mL) | (ng/mL) | (ng/mL) | (ng/mL) | (ng/mL) | CV | CI |
| 1 | 4 | 93.2 | 25.6 | 32.6 | 282.0 | 125.9 | 135% | 123.4 |
| 45 | 3 | 23.1 | 12.9 | 15.1 | 41.3 | 15.8 | 68% | 17.9 |
| 90 | 3 | 52.1 | 20.3 | 26.0 | 110.0 | 50.2 | 96% | 56.8 |
| 137 | 4 | 115.8 | 58.4 | 89.8 | 225.0 | 77.6 | 67% | 76.0 |
| 180 | 4 | 296.0 | 85.0 | 264.0 | 571.0 | 209.8 | 71% | 205.6 |
| 225 | 3 | 184.9 | 2.9 | 21.7 | 530.0 | 299.0 | 162% | 338.4 |
| 270 | 3 | 30.0 | 2.9 | 30.8 | 56.2 | 26.7 | 89% | 30.2 |

^{A} The method for the determination of soluble axitinib in VH had a LLOQ = 0.100 ng/mL

Median axitinib concentrations of axitinib in rabbit retina samples over the study duration (see Table 3) were maximal (206.0 ng/g) at 137 days. Individual samples ranged from a minimum of 9.6 ng/g at study termination (day 270) to a maximum of 522.1 ng/g (day 180). Median axitinib concentrations in the retina were similar between days 1 through 180, and prior to a noted decrease down to 14.6 ng/g at day 225. This indicates rapid and sustained transport of axitinib to the targeted retina tissues from hydrogel implant within 1 day of administration through approximately 6 months. There was a noted waning that was approximately 10× less (147.1 to 14.6 ng/g) in the axitinib concentrations from day 180 to day 225 in the retinal tissue samples.

TABLE 3
Summary of Axitinib Concentrations in Retina^{A}

| | | Average | Min | Median | Max | Std Dev | | 95% |
|---|---|---|---|---|---|---|---|---|
| Day | N | (ng/g) | (ng/g) | (ng/g) | (ng/g) | (ng/g) | CV | CI |
| 1 | 4 | 184.7 | 116.0 | 147.4 | 328.1 | 97.7 | 53% | 95.7 |
| 45 | 3 | 165.5 | 69.0 | 169.9 | 257.6 | 94.4 | 57% | 106.8 |
| 90 | 3 | 176.8 | 120.2 | 203.3 | 207.0 | 49.1 | 28% | 55.5 |
| 137 | 4 | 271.8 | 153.0 | 206.0 | 522.1 | 170.3 | 63% | 166.9 |
| 180 | 4 | 150.0 | 18.8 | 147.1 | 287.0 | 120.7 | 80% | 118.2 |
| 225 | 3 | 15.3 | 13.6 | 14.6 | 17.7 | 2.1 | 14% | 2.4 |
| 270 | 3 | 13.6 | 9.6 | 10.3 | 20.8 | 6.3 | 46% | 7.1 |

^{A}The method for the determination of axitinib in retina had a LLOQ = 0.100 ng/mL

Median axitinib concentrations of axitinib in rabbit choroid samples over the study duration (see Table 4) were maximal (306.5 ng/g) at 190 days. Individual samples ranged from a minimum of 15.2 ng/g at study termination (day 270) to a maximum of 581.6 ng/g (day 90). Median axitinib concentrations in the choroid were similar between days 1 through 180, and prior to a noted decrease down to 33.3 ng/g at day 225. This indicates rapid and sustained transport of axitinib to the choroid tissues from hydrogel implant within 1 day of administration through approximately 6 months. There was a noted waning that was approximately 3× less (98.4 to 33.3 ng/g) in the axitinib concentrations from day 180 to day 225 in the choroid tissue samples.

TABLE 4
Summary of Axitinib Concentrations in Choroid ^{A}

| | | Average | Min | Median | Max | Std Dev | | 95% |
|---|---|---|---|---|---|---|---|---|
| Day | N | (ng/g) | (ng/g) | (ng/g) | (ng/g) | (ng/g) | CV | CI |
| 1 | 4 | 124.3 | 78.5 | 119.6 | 179.6 | 48.4 | 39% | 47.5 |
| 45 | 3 | 256.6 | 128.1 | 278.7 | 363.0 | 119.0 | 46% | 134.7 |
| 90 | 3 | 328.2 | 96.6 | 306.5 | 581.6 | 243.2 | 74% | 275.2 |
| 137 | 4 | 283.3 | 188.8 | 232.4 | 479.5 | 133.1 | 47% | 130.5 |
| 180 | 4 | 95.0 | 52.0 | 98.4 | 131.0 | 32.6 | 34% | 31.9 |
| 225 | 3 | 35.0 | 18.7 | 33.3 | 52.9 | 17.2 | 49% | 19.4 |
| 270 | 3 | 34.8 | 15.2 | 22.8 | 66.3 | 27.6 | 79% | 31.2 |

^{A} The method for the determination of axitinib in retina had a LLOQ = 0.100 ng/mL for days 1, 45, 90 and 137 and a LLOQ = 0.200 ng/mL for days 180, 225 and 270

The solubility of axitinib measured (internal communication) in vitro in

biorelevant media under physiological conditions (PBS, pH 7.2 at 37° C.) is 540 ng/mL. This solubility value is similar to that observed for the maximum individual sample concentrations of axitinib in the soluble VH (571.0 ng/ml), retina (522.1 ng/g) and choroid (581.6 ng/g). If axitinib was accumulating over the study duration in these tissues, then maximal values might be expected to be higher than the solubility observed in vitro in biorelevant media.

The hydrogel implant test articles were formulated to contain approximately 110 μg of axitinib drug substance per implant. The measurement of non-soluble (undissolved) axitinib in the VH containing the implant at different timepoints allows an assessment of axitinib released from hydrogel implant over time, Table 5. Results demonstrate that 90.0 μg of axitinib (109.4 μg minus 19.4 μg) was released over 180 days. This amount released over 180 days is similar to that observed in a the hydrogel implantocular distribution study in beagle dogs using the same hydrogel implant test articles. Assuming a consistent release over 6 months, based on the consistent retina tissue concentrations over that time period, then this equates to a daily axitinib release rate of approximately 0.5 μg per day from hydrogel implant. The rate of axitinib release from inventive implant slows down from days 180 to study completion and this may be due to the near complete biodegradation of the hydrogel which occurs in DB rabbits between 5 and 6 months leading to a localization in the VH of the undissolved axitinib.

TABLE 5
Summary of Non-Soluble Axitinib in
VH Containing Hydrogel Implant^{A}

| | | Average | Min | Median | Max | Std Dev | | 95% |
|---|---|---|---|---|---|---|---|---|
| Day | N | (μg) | (μg) | (μg) | (μg) | (μg) | CV | CI |
| 1 | Excluded from Analysis ^{B} | | | | | | | |
| 45 | | | | | | | | |
| 90 | | | | | | | | |
| 137 | | | | | | | | |
| 180 | 4 | 19.6 | 9.2 | 19.4 | 30.3 | 8.6 | 44% | 8.4 |
| 225 | 3 | 19.0 | 12.4 | 12.7 | 31.9 | 11.2 | 59% | 12.6 |
| 270 | 3 | 11.0 | 2.4 | 10.9 | 19.6 | 8.6 | 79% | 9.8 |

^{A}The method for the determination of non-soluble axitinib in VH containing hydrogel implant had a LLOQ = 0.100 ng/mL.
^{B} All non-soluble axitinib in the VH containing hydrogel implant samples from days 1, 45, 90 and 137 were excluded from analysis due to incomplete drug extraction and the method was rectified with an improved extraction procedure for the samples from days 180, 225 and 270.

### CONCLUSIONS

The inventive hydrogel test articles contained approximately 109 μg of axitinib and hydrated to an approximate dimension of 0.5 mm diameter by 9.2 mm length in biorelevant media.

Plasma concentrations of axitinib for all DB rabbit samples are <LLOQ (0.0500 ng/mL) indicating near absent systemic exposure to axitinib in the rabbit model following hydrogel implant administration.

Concentrations of axitinib in rabbit AH samples over the study duration were low relative to the other ocular tissues indicating little axitinib migration towards the anterior chamber from the posterior chamber. Median axitinib concentrations in the retina and choroid were elevated (between 98.4 to 306.5 ng/g) within 1 day of administration through approximately 6 months indicating rapid and sustained transport of axitinib to the targeted tissues from hydrogel implant and then waned in the subsequent 3 months.

The maximum concentrations observed in individual samples in the VH, retina and choroid were similar to the maximal solubility in vitro in biorelevant media, indicating no apparent accumulation of axitinib in the tissues over the study duration.

After the hydrogel degrades and disappears at about 4.5 months, residual undissolved drug was observed to remain in the vitreous humor fluid, having condensed to an aggregated mass. Although axitinib concentrations in the vitreous humor remained high at 180 and 225 days, concentrations in the retina and choroid declined after 137 days, roughly corresponding with the disappearance of the hydrogel. This indicates that the hydrogel aids in maintaining a faster rate of drug release by preventing aggregation of drug particles into a condensed mass.

### C Safety, Tolerability, and Activity in Humans

Subjects with neovascular age-related macular degeneration (nAMD, both treatment-naïve and those with a history of anti-VEGF therapy) were enrolled for administration of inventive hydrogel in a single study eye. Two groups completed enrollment and are under evaluation: 200 μg axtinib in a 7.5% PEG hydrogel (formed from 2 parts 4a20K PEG-SAZ to 1 parts 8a20K PEG amine) where the 7.5% represents the PEG weight divided by the fluid weight×100 (1 implant; n=6) and 400 μg axtinib (2 implants; n=7). Spectral-domain optical coherence tomography (SD-OCT) imaging was used to assess retinal fluid and central subfield thickness (CSFT) was performed at Baseline. Injection visits occurred at days 3, 7, and 14, and at months 1, 2, 3, 4.5, 6, 7.5, 9, and, approximately monthly until implant(s) were no longer visible. The inventive implants were visualized at every visit. Safety evaluations include: adverse event collection, vital signs, best-corrected visual acuity (BCVA), slit lamp biomicroscopy, tonometry, indirect and direct ophthalmoscopy and safety labs.

In the 400 μg group, an average reduction in central subfield thickness (CSFT) of 89.8+22.5 μm (mean+SEM) was observed by 2 months and was generally maintained through the 3 month timepoint (follow-up ongoing). For several subjects with a history of anti-VEGF therapy, the durability of anti-VEGF treatment was extended to >9 months in the 200 μg group and >3 months in the 400 μg group (follow-up ongoing). Best-corrected visual acuity (BCVA) was maintained with no serious ocular adverse events reported. The most common adverse events observed in the study eye include tiny pigmented keratic precipitates (3/13), subretinal hemorrhage (2/13) and subconjunctival hemorrhage (3/13) and pain (2/13) following implant injection. Implant(s) exhibited little movement in the vitreous and were no longer visible after 9-10.5 months in the 200 μg group.

The inventive implants were generally well-tolerated with a favorable safety profile. Minimal movement and consistent resorption of implant(s) has been observed up to 10.5 months.

While we have described a number of embodiments of this, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

1. A system comprising (i) a sustained-release biodegradable ocular hydrogel implant comprising a tyrosine kinase inhibitor and a polymer network, wherein the ocular hydrogel implant after ocular administration comprises and a clearance zone, wherein the clearance zone is devoid of undissolved TKI particles prior to release of the TKI and (ii) a needle that is tipped with annealed polyethylene glycol, wherein the implant is loaded into the needle.

2. The system of claim 1, wherein the ocular hydrogel implant is in the form of a cut strand prior to administration.

3. The system of claim 1, wherein the dissolved TKI is present in the hydrogel implant at or near its saturation level.

4. The system of claim 1, wherein the size of the clearance zone increases as a function of the amount of TKI release.

5. The system claim 1, wherein the ocular hydrogel implant is fully degraded following release of at least 90% of the TKI.

6. The system claim 1, wherein the ocular hydrogel implant is fully degraded after about 3 months following complete release of the TKI.

7. The system of claim 1, wherein degradation of the ocular hydrogel occurs prior to release of the TKI.

8. The system of claim 1, wherein the polymer network comprises a plurality of polyethylene glycol (PEG) units.

9. The system of claim 1, wherein the polymer network comprises a plurality of multi-arm PEG units.

10. The system of claim 1, wherein the polymer network comprises a plurality of 4- or 8-arm PEG units.

11. The system of claim 1, wherein the polymer network comprises a plurality of PEG units having the formula: 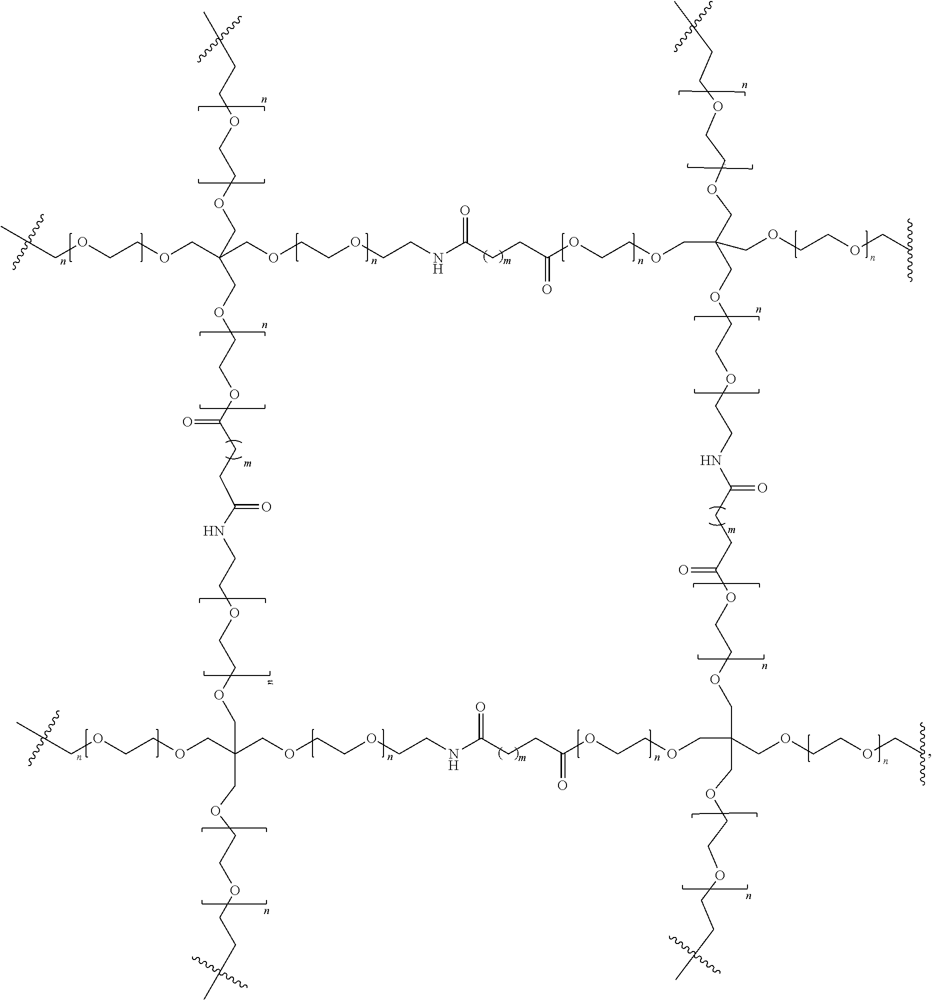
wherein n represents an ethylene oxide repeating unit and the wavy lines represent the points of repeating units of the polymer network.

12. The system of claim 1, wherein the polymer network is formed by reacting a plurality of polyethylene glycol (PEG) units selected from 4a20K PEG SAZ, 4a20K PEG SAP, 4a20K PEG SG, 4a20K PEG SS, 8a20K PEG SAZ, 8a20K PEG SAP, 8a20K PEG SG, and 8a20K PEG SS with one or more PEG or Lysine based-amine groups selected from 4a20K PEG NH2, 8a20K PEG NH2, and trilysine, or a salt thereof.

13-15. (canceled)

16. The system of claim 1, wherein the tyrosine kinase inhibitor is homogenously dispersed within the polymer network.

17. The system of claim 1, wherein the tyrosine kinase inhibitor is released over a period of at least 15 days.

18. The system of claim 1, wherein the tyrosine kinase inhibitor is released over a period of at least 30 days.

19. The system of claim 1, wherein the tyrosine kinase inhibitor is released over a period of at least 60 days.

20. The system claim 1, wherein the tyrosine kinase inhibitor is released over a period of at least 90 days.

21-24. (canceled)

25. The system of claim 1, wherein the tyrosine kinase inhibitor is selected from abemaciclib, acalabrutinib, afatinib, alectinib, axitinib, barictinib, binimetinib, brigatinib, cabozantinib, ceritinib, coblmetinib, crizotinib, dabrafenib, dacomitinib, dasatinib, encorafenib, erlotinib, everolimus, fostamatinib, gefitinib, gilteritinib, ibrutinib, imatinib, larotrectinib, lenvatinib, lorlatinib, axitinib, idelalisib, lenvatinib, midostaurin, neratinib, netarsudil, nilotinib, nintedanib, osimertinib, palbociclib, pazopanib, ponatinib, regorafenib, ribociclib, ruxolitinib, sirolimus, sorafenib, sunitinib, temsirolimus, tofacitinib, trametinib, vandetanib, and vemurafenib.

26. The system of claim 1, wherein the tyrosine kinase inhibitor is axitinib.

27. (canceled)

28. A method of treating an ocular condition in a subject in need thereof, comprising utilizing the system of claim 1 for injecting or affixing the ocular hydrogel implant to the subject.

29-32. (canceled)
